(19) United States
(12) Patent Application Publication
(10) Pub. No.: US 2015/0166436 A1
(43) Pub. Date: Jun. 18, 2015

(54) PROCESSES AND APPARATUSES FOR PREPARING AROMATIC COMPOUNDS

(71) Applicant: UOP LLC, Des Plaines, IL (US)

(72) Inventor: Jason L. Noe, Mount Prospect, IL (US)

(73) Assignee: UOP LLC, Des Plaines, IL (US)

(21) Appl. No.: 14/104,808

(22) Filed: Dec. 12, 2013

(51) Int. Cl. C07C 5/03; C07C 5/13

(52) U.S. Cl. CPC C07C 5/03; C07C 5/13

(57) ABSTRACT

Processes and apparatuses for preparing aromatic compounds are provided. In an embodiment, a process of preparing aromatic compounds includes providing a heavy reformate stream including C7 hydrocarbons, xylenes, styrene, and compounds having more than 8 carbon atoms. The xylenes and styrene are separated from the compounds that have more than 8 carbon atoms in the heavy reformate stream to form a mixed xylene stream including the xylenes and styrene and a C9+ stream including compounds with less volatility than C8 aromatics. The styrene within the mixed xylene stream is selectively hydrogenated to form a hydrogenated xylene stream including xylenes and ethylbenzene. At least some of the xylenes are separated from the ethylbenzene in the hydrogenated xylene stream to form a C8 raffinate stream including the ethylbenzene and a xylene product stream including one or more xylene species.

### TECHNICAL FIELD

The technical field generally relates to processes and apparatuses for preparing aromatic compounds, and more particularly relates to processes and apparatuses for preparing aromatic compounds with efficient treatment of olefins and alkenyl benzene during processing.

### BACKGROUND

Aromatic compounds have a multitude of uses, both as end products and as reactants for downstream processes. Methods of preparing aromatic compounds from a hydrocarbon feed are generally known in the art and include upgrading the hydrocarbon feed followed by reforming and aromatics separation. Typical upgrading techniques include hydrotreating to remove contaminants such as sulfur, nitrogen, and oxygen. After upgrading, the hydrocarbon feed is reformed in the presence of a catalyst to convert paraffins and naphthenes to a reformate that includes aromatic compounds such as xylenes, benzene, and toluene. A series of separation techniques are employed to separate the various aromatic compounds from the reformate, and numerous product streams having varying degrees of purity may be isolated for each aromatic compound in the reformate.

The presence of olefins and other unsaturated compounds within the reformate is problematic during various unit operations that are employed to process or separate various compounds from the reformate. For example, transalkylation is a common unit operation conducted to convert alkylated aromatic compounds in a mixture that is significantly different from an equilibrium mixture to a mixture that is much closer to equilibrium in the presence of a transalkylation catalyst under transalkylating conditions, with toluene, C9, and heavier compounds generally subject to transalkylation to increase benzene and xylene yields. A side reaction of most transalkylation units is the cracking of ethyl and higher alkyl groups from the rings of C9 and heavier aromatic compounds, which leads to a mixture of primary monoalkylated aromatic compounds such as toluene and xylenes. Conventional transalkylation catalysts are sensitive to the presence of olefins and alkenyl benzene in the feed that is subject to transalkylation such that olefins and alkenyl benzene are generally removed or converted to saturated species prior to conventional transalkylation. Further, recovery of xylenes is generally conducted by first separating xylenes and compounds having at least 8 carbon atoms (and oftentimes compounds having 7 carbon atoms as well) from the reformate in a reformate splitter, followed by separating xylenes and compounds having 8 (and, when present, 7) carbon atoms from compounds having greater than 8 carbon atoms in a xylene column. The xylenes and compounds having 7 and 8 carbon atoms are then subject to adsorptive separation to separate various xylene isomers. However, adsorption beds that are generally employed to separate the xylene isomers are sensitive to the presence of unsaturated compounds, such as styrene, such that olefins and alkenyl benzene are generally removed or converted to saturated species prior to adsorptive separation to separate the various xylene isomers.

Existing techniques for removing or converting olefins and alkenyl benzene include olefin reduction processing (ORP) and clay treatment. ORP selectively hydrogenates olefins and alkenyl benzene in the presence of a catalyst but is generally less effective for unsaturated compounds that exhibit significant steric hinderance of the unsaturated functionality, with particular concern for species having over 10 carbon atoms. Further, ORP can increase operating cost and capital investment, often requiring a stripping column to remove excess hydrogen from the process especially when ORP is conducted on streams that have a high bromine index (which corresponds to high content of olefins and alkenyl benzene). Clay treatment is effective to remove all olefins but results in spent clay that requires remediation, and further results in loss of product yield due to conversion of olefins to compounds that cannot be recovered during processing. In particular, during clay treatment, unsaturated compounds react with aromatic compounds in the presence of the clay to produce combined molecules that are ultimately removed from the process as a low value heavy oil and contribute to product yield loss. Additionally the combined molecules increase the required temperatures for downstream fractionation, which adds to processing cost. For example, unsaturated aromatic compounds such as methyl styrene react with other aromatic compounds in the presence of the clay to produce biphenyl compounds, which are ultimately removed as a heavy oil and decrease product yield from the initial hydrocarbon feed. Due to the various unit operations that require olefins and alkenyl benzene in feed streams to be treated prior thereto, multiple ORP and/or clay treatment unit operations are generally employed at various locations within the process.

Accordingly, it is desirable to provide novel processes and apparatuses for preparing aromatic compounds that enable efficient treatment of olefins and alkenyl benzene while minimizing loss of product yield and while further avoiding detrimental effects of olefins and alkenyl benzene on production and separation of various aromatic products. Furthermore, other desirable features and characteristics of the present invention will become apparent from the subsequent detailed description of the invention and the appended claims, taken in conjunction with the accompanying drawings and this background of the invention.

### BRIEF SUMMARY

Processes and apparatuses for preparing aromatic compounds are provided herein. In an embodiment, a process of preparing aromatic compounds includes providing a heavy reformate stream that includes C7 hydrocarbons, xylenes, styrene, and compounds having more than 8 carbon atoms. Xylenes and styrene are separated from the compounds that have more than 8 carbon atoms in the heavy reformate stream to form a mixed xylene stream that includes the xylenes and styrene and a C9+stream that includes compounds with less volatility than C8 aromatics. The styrene within the mixed xylene stream is selectively hydrogenated to form a hydrogenated xylene stream that includes xylenes and ethylbenzene. At least some of the xylenes are separated from the ethylbenzene in the hydrogenated xylene stream to form a C8 raffinate stream that includes the ethylbenzene and a xylene product stream that includes one or more xylene species.

In another embodiment, a process for preparing aromatic compounds includes hydrotreating a naphtha feed stream to form a hydrotreated stream. The hydrotreated stream is reformed to produce a reformate stream. The reformate stream is fractionated into a heavy reformate stream that includes C7 hydrocarbons, xylenes, styrene, and compounds having more than 8 carbon atoms and a light reformate overhead stream that includes compounds having higher volatility than xylene. Aromatic compounds are separated from non-aromatic compounds in the light reformate overhead stream to produce a reformate aromatic stream and a reformate raffinate stream. The xylenes and styrene are separated from the compounds having more than 8 carbon atoms in the heavy reformate stream to form a mixed xylene stream that includes the xylenes and styrene and a C9+ stream that includes compounds with less volatility than C8 aromatics. The styrene within the mixed xylene stream is selectively hydrogenated to form a hydrogenated xylene stream that includes xylenes and ethylbenzene. The hydrogenated xylene stream is separated into a xylene product stream that includes para-xylene, a meta-xylene product stream that includes meta-xylene, and a meta-xylene raffinate stream that includes ethylbenzene. In another embodiment, an apparatus for preparing aromatic compounds includes a hydrotreating unit for receiving a naphtha feed stream and for hydrotreating the naphtha feed stream to form a hydrotreated stream. A reforming unit is in fluid communication with the hydrotreating unit for receiving the hydrotreated stream and for reforming the hydrotreated stream to produce a reformate stream. A reformate splitter is in fluid communication with the reforming unit for receiving the reformate stream and for fractionating the reformate stream into a heavy reformate stream that includes C7 hydrocarbons, xylenes, styrene, and compounds having more than 8 carbon atoms and a light reformate overhead stream that includes compounds having higher volatility than xylene. A xylene column is in fluid communication with the reformate splitter for receiving and for separating the heavy reformate stream to produce a mixed xylene stream that includes xylenes and styrene and a C9+ stream that includes compounds with less volatility than C8 aromatics. An olefin reduction processing unit is in fluid communication with the xylene column for receiving the mixed xylene stream and for selectively hydrogenating the styrene within the mixed xylene stream to form a hydrogenated xylene stream that includes xylenes and ethylbenzene. An adsorption unit is in fluid communication with the olefin reduction processing unit for receiving the hydrogenated xylene stream and for separating xylene from the ethylbenzene in the hydrogenated xylene stream to form a C8 raffinate stream that includes the ethylbenzene and a xylene product stream that includes one or more xylene species. An isomerization unit is in fluid communication with the adsorption unit for isomerizing the ethylbenzene from the C8 raffinate stream in the presence of an isomerization catalyst to produce an isomerized aromatic stream comprising xylenes.

### BRIEF DESCRIPTION OF THE DRAWINGS

The various embodiments will hereinafter be described in conjunction with the following drawing figures, wherein like numerals denote like elements, and wherein:

FIG. 1 is a schematic diagram of an apparatus and a method for preparing aromatic compounds in accordance with an exemplary embodiment.

### DETAILED DESCRIPTION

The following detailed description is merely exemplary in nature and is not intended to limit the various embodiments or the application and uses thereof. Furthermore, there is no intention to be bound by any theory presented in the preceding background or the following detailed description.

Processes and apparatuses for preparing aromatic compounds are provided herein that enable olefins and alkenyl benzene in a heavy reformate stream to be efficiently treated to avoid adverse effects associated with the presence of the olefins and alkenyl benzene during processing while maximizing product yield. In particular, styrene and other unsaturated compounds that may be present along with xylenes are selectively hydrogenated after separating xylenes and styrene (and other compounds that may be present in a heavy reformate stream, as described in further detail below) from compounds that have more than 8 carbon atoms in the heavy reformate stream, thereby avoiding adverse effects of olefins and alkenyl benzene on further separation of the xylenes. Because a content of olefins and alkenyl benzene (e.g., styrene) is generally low after separating xylenes and styrene from compounds that have more than 8 carbon atoms in the heavy reformate stream, minimal amounts of hydrogen gas are needed to convert the olefins and alkenyl benzene to their saturated analogs (e.g. styrene is converted to saturated ethylbenzene) such that a separate stripper is not required to remove excess hydrogen. Additionally, transalkylation of a stream containing compounds having less than 8 carbon atoms, e.g., toluene, is conducted in the presence of a catalyst that includes acid function and metal function, which has been found to effectively function in the presence of olefins and alkenyl benzene unlike conventional transalkylating catalysts such that treatment of olefins and alkenyl benzene prior to transalkylation is not required and treatment of olefins and alkenyl benzene prior to separation of the xylenes is sufficient to avoid adverse effects of olefins and alkenyl benzene within the processes and apparatuses while maximizing process efficiency.

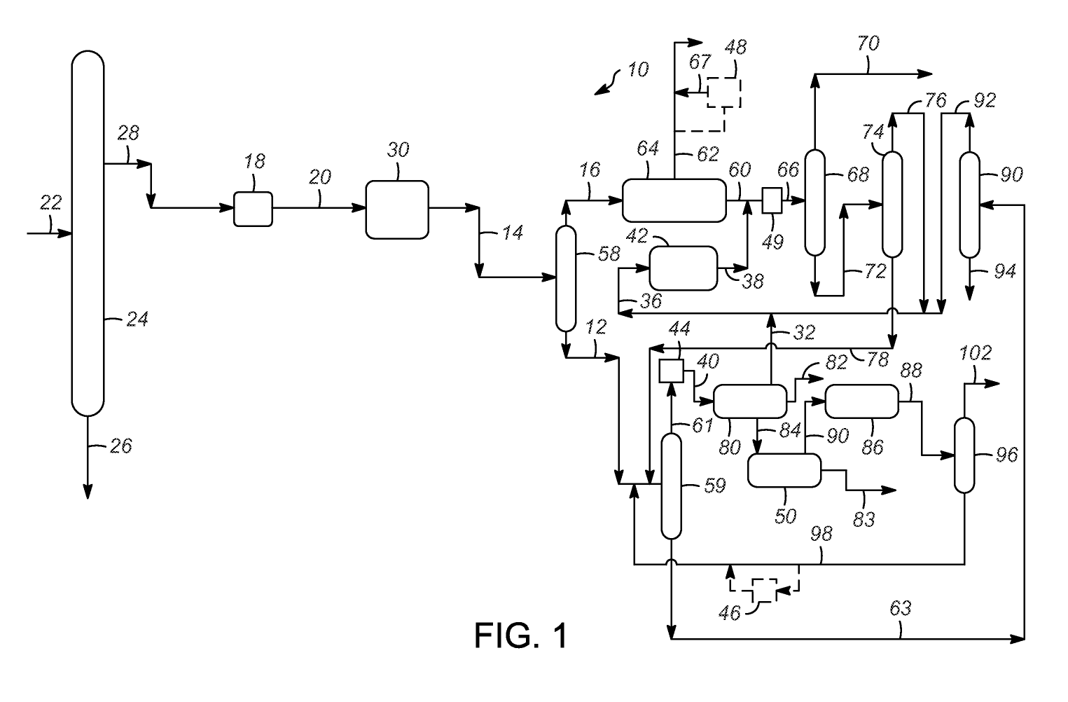

An embodiment of a process for preparing aromatic compounds will now be described with reference to an exemplary apparatus 10 for producing aromatic compounds as shown in FIG. 1. In accordance with the process and as shown in FIG. 1, a heavy reformate stream 12 is provided that includes C7 hydrocarbons, xylenes, styrene, and compounds having more than 8 carbon atoms. Ethylbenzene may also be present in the heavy reformate stream 12. As referred to herein, the “heavy reformate stream” is a hydrocarbon stream that is produced by fractionation of a reformate stream 14 that is produced through catalytic reforming, as described in further detail below, and that contains saturated aromatic compounds having at least 7 carbon atoms along with other carbon-containing compounds that have a boiling point of at least that of saturated C7 aromatic compounds. It is to be appreciated that the heavy reformate stream 12 may include additional components beyond those recited above, although it is also to be appreciated that unsaturated cyclic, linear, or branched C7 hydrocarbons are generally absent from the heavy reformate stream 12. In particular, unsaturated cyclic, linear, and branched non-aromatic species that tend to boil with aromatics are typically one carbon number higher until the alkenyl benzene species, then the carbon numbers come back together with similar boiling points for the aromatics and the unsaturated cyclic, linear, and branched non-aromatic species. Styrene, which is a C8 unsaturated alkenyl benzene, boils with the C8 aromatics: ethylbenzene and the xylenes, although 1-octene, which is a C8 unsaturate, coboils with toluene. As such, the heavy reformate stream 12 generally includes saturated aromatic compounds that have 7 carbon atoms, such as toluene, as well as saturated or unsaturated cyclic, linear, or branched C8/C9 compounds, while unsaturated C7 hydrocarbons are generally absent from the heavy reformate stream 12. The phrase “C8/C9 compounds”, and similar designations, is intended to encompass any compound or grouping of compounds that have the specified number of carbon atoms, and includes aromatic or aliphatic compounds as well as compounds that contain any heteroatoms unless indicated otherwise.

A source of the heavy reformate stream 12 is not particularly limited provided that the heavy reformate stream 12 includes the recited components and originates from fractionation of the reformate stream 14 that is produced through catalytic reforming In an embodiment, the heavy reformate stream 12 is derived from distillation of a crude carbonaceous feed 22 that produces a naphtha feed stream 28, followed by hydrotreating the naphtha feed stream 28 to form a hydrotreated stream 20, reforming the hydrotreated stream 20 to produce the reformate stream 14, and then fractionating the reformate stream 14 into the heavy reformate stream 12 and a light reformate overhead stream 16 that includes compounds having higher volatility than xylene. Distillation of the crude carbonaceous feed 22 may be conducted in a crude distillation unit 24 to produce the naphtha feed stream 28, along with a crude heavy stream 26 that may be separately processed through conventional techniques not described herein. The naphtha feed stream 28 is hydrotreated for purposes of removing sulfur and other impurity species that may be present in the naphtha feed stream 28 after crude distillation. In an embodiment and as shown in FIG. 1, a hydrotreating unit 18 is in fluid communication with the crude distillation unit 24 for receiving the naphtha feed stream 28 and for hydrotreating the naphtha feed stream 28 to produce the hydrotreated stream 20. The naphtha feed stream 28 generally includes compounds that have from 6 to 11 carbon atoms. Hydrotreating may be conducted through conventional techniques to produce the hydrotreated stream 20.

After hydrotreating, the hydrotreated stream 20 is reformed to produce the reformate stream 14. In particular, in an embodiment, the hydrotreated stream 20 is catalytically reformed in the presence of a platinum- and/or rhenium-containing catalyst to produce the reformate stream 14, which includes paraffins, olefins, napthenes and aromatic components. In an embodiment and as shown in FIG. 1, a reforming unit 30 is in fluid communication with the hydrotreating unit 18 for receiving the hydrotreated stream 20 and for reforming the hydrotreated stream 20 to produce the reformate stream 14. To reform the hydrotreated stream 20, the hydrotreated stream 20 may be mixed with hydrogen, followed by contacting the resulting stream with the platinum- and/or rhenium-containing catalyst to convert paraffins and naphthenes in the hydrotreated stream 20 to aromatic compounds through dehydrogenation and cyclization. Reforming may be conducted under conventional conditions, and conventional platinum- and/or rhenium-containing catalysts may be employed.

Aromatic compounds that are produced through reforming generally include benzene, toluene, and xylenes, all of which may be useful end products for various applications and which may be separated through various unit operations. In accordance with an embodiment, the reformate stream 14 is fractionated to produce the heavy reformate stream 12 that includes at least C7 hydrocarbons (e.g., toluene), xylenes, styrene, and compounds having more than 8 carbon atoms, as set forth above, and a light reformate overhead stream 16 that includes compounds having higher volatility than xylene (e.g., toluene, non-aromatic co-boilers of toluene that are predominantly C8 hydrocarbons, and any hydrocarbons having higher volatility than ethylbenzene that remain in the reformate stream 14 after reforming) Thus, toluene may be present in both the heavy reformate stream 12 and the light reformate overhead stream 16. To conduct fractionation of the reformate stream 14, a reformate splitter 58 may be in fluid communication with the reforming unit 30 for receiving the reformate stream 14 and for fractionating the reformate stream 14.

The heavy reformate stream 12 is subject to further processing for xylene recovery. In an embodiment, the xylenes and styrene, as well as any ethylbenzene that is present, are separated from the compounds with less volatility than the C8 aromatics in the heavy reformate stream 12 to form a mixed xylene stream 61 that includes the xylenes and styrene, as well as some compounds that may be present in the heavy reformate stream 12 with volatility less than or equal to that of the xylene species, and a C9+ stream 63 that includes compounds with less volatility than C8 aromatics (e.g., compounds that generally have more than 8 carbon atoms). While it is to be appreciated that the mixed xylene stream 61 and the C9+ stream 63 may include some compounds that do not precisely fit within the aforementioned values, the respective streams include a majority of compounds having the specified description consistent with yields that are realized through conventional fractionation, and this applies to all references herein to various streams that have compounds with a specified composition and that are obtained through fractionation or distillation. In an embodiment and as shown in FIG. 1, a xylene column 59 may be in fluid communication with the reformate splitter 58 for receiving and for separating the heavy reformate stream 12 into the mixed xylene stream 61 and the C9+ stream 63 through conventional fractionation techniques.

In an embodiment, the xylenes and styrene are separated from the compounds with less volatility than the C8 aromatics in the heavy reformate stream 12 in the absence of selective hydrogenation of the heavy reformate stream 12. In particular, in this embodiment, the reformate splitter 58 and the xylene column 59 are in fluid communication in the absence of a clay unit or a hydrogenation unit, such as an olefin reduction processing (ORP) unit, therebetween such that styrene remains in the mixed xylene stream 61. By avoiding selective hydrogenating of the heavy reformate stream 12, unnecessary treatment of olefins and alkenyl benzene that are separated into the C9+ stream 63 is avoided, thereby minimizing loads on treatment of olefins and alkenyl benzene within the process.

The styrene and any other olefins and alkenyl benzene within the mixed xylene stream 61 are selectively hydrogenated to form a hydrogenated xylene stream 40 that includes xylenes and ethylbenzene, followed by separating xylene from the ethylbenzene Selective hydrogenation of the mixed xylene stream 61 is conducted to minimize the presence of olefins and alkenyl benzene therein, which may have a detrimental impact on downstream separation of xylene through adsorption. In this regard, selective hydrogenation is conducted prior to separation of the xylene from the hydrogenated xylene stream 40, either immediately prior thereto (i.e., with no other unit operations conducted between selective hydrogenation and separation of xylene from the hydrogenated xylene stream 40) or with other optional unit operations between selective hydrogenation and separation of the xylene from the hydrogenated xylene stream. Selective hydrogenation may be conducted through olefin reduction processing, which converts the olefins and alkenyl benzene including styrene to a corresponding saturated species (e.g., ethylbenzene for styrene) in the presence of hydrogen and an appropriate catalyst. While olefin reduction processing is generally less effective for unsaturated compounds that exhibit steric hinderance of the unsaturated functionality and/or as the carbon number of the species increases (which becomes more of a consideration over 10 carbon atoms), the mixed xylene stream 61 generally only includes C8 and, in various embodiments, some C9 compounds. Because unsaturated C8 and C9 compounds are readily converted to their corresponding saturated species through olefin reduction processing, olefin reduction processing of the mixed xylene stream 61 is efficient and effective to minimize the presence of olefins and alkenyl benzene in the hydrogenated mixed xylene stream 40. Olefin reduction processing may be conducted, for example, in an ORP unit 44 that is in fluid communication with the xylene column 59 for receiving the mixed xylene stream 61 and for selectively hydrogenating the styrene within the mixed xylene stream 61 to form the hydrogenated xylene stream 40. The ORP unit 44 may include an appropriate catalyst, such as a nickel on alumina catalyst, and may be operated under conventional operating conditions.

The mixed xylene stream 61, and the hydrogenated xylene stream 40 after selective hydrogenation, generally includes various C8 aromatic isomers, such as ethylbenzene, para-xylene, meta-xylene, and/or ortho-xylene, and the various isomers in the hydrogenated xylene stream 40 may be further processed for C8 aromatic isomer separation after selective hydrogenation. At least some of the xylene is separated from the hydrogenated xylene stream 40 to form a xylene product stream 82 that includes one or more xylene species and a C8 raffinate stream 84 that includes ethylbenzene, among other compounds. The “xylene product stream”, as referred to herein, includes one or more xylene species (such as predominantly para-xylene, in an embodiment) that is taken as product and is not subject to further processing in accordance with the methods described herein. The “C8 raffinate stream”, as referred to herein, includes non-para-xylene C8 compounds such as the ethylbenzene, and may optionally include meta-xylene, ortho-xylene, as well as any C9 compounds that may be present in the hydrogenated mixed stream 40. Para-xylene is generally a more commercially valuable xylene isomer than other xylene isomers and, thus, is generally separated from the other C8 aromatic isomers through conventional separation techniques. In an embodiment and as shown in FIG. 1, the para-xylene isomer is separated from the other C8 aromatic isomers in the hydrogenated xylene stream 40 to form a crude toluene stream 32, a xylene product stream 82 that includes para-xylene, and the C8 raffinate stream 84. The “crude toluene stream”, as referred to herein, includes C7 aromatic compounds, e.g., toluene, that may be present in the hydrogenated xylene stream 40 and some para-xylene. In an embodiment, and as shown in FIG. 1, an adsorption unit 80 is in fluid communication with the ORP unit 44 for receiving the hydrogenated xylene stream 61 and for separating xylenes from the ethylbenzene in the hydrogenated xylene stream 40 through adsorption/desorption to form the C8 raffinate stream 84 and the xylene product stream 82. In particular, in the embodiment shown in FIG. 1, the adsorption unit 80 separates the hydrogenated xylene stream 40 into the xylene product stream 82 that includes para-xylene, the C8 raffinate stream 84 that includes ethylbenzene, meta-xylene, and ortho-xylene, and the crude toluene stream 32 that includes C7 aromatic compounds and some para-xylene. In embodiments and although not shown, ortho-xylene may be separated from the heavy reformate stream 12 into the C9+ stream 63, and further separated from C9+ compounds to produce an isolated ortho-xylene stream. In this embodiment, ortho-xylene may not be present in the C8 raffinate stream 84, beyond trace amounts.

In an embodiment and as shown in FIG. 1, the C8 raffinate stream 84 is separated into a meta-xylene product stream 83 that includes meta-xylene and a meta-xylene raffinate stream 90. In this embodiment, a meta-xylene adsorption unit 50 may be in fluid communication with the adsorption unit 80 for receiving the C8 raffinate stream 84 and for separating the C8 raffinate stream 84 into the meta-xylene product stream 83 and the meta-xylene raffinate stream 90, which may include ortho-xylene and ethylbenzene. The meta-xylene raffinate stream 90 may be further isomerized through conventional techniques in an isomerization unit 86 that is in fluid communication with the meta-xylene adsorption unit 50, thereby isomerizing some of the ortho-xylenes to para-xylenes in the presence of an isomerization catalyst and producing an isomerized aromatic stream 88 that includes xylenes. The isomerization unit 86 also generally isomerizes and dealkylates the ethylbenzene to benzene and ethane, and isomerization catalysts employed in the isomerization unit generally include both metal and acid function.

It is to be appreciated that in embodiments and although not shown, the meta-xylene adsorption unit 50 may be omitted and the C8 raffinate stream 84 may be provided directly for further isomerization, in which case both ortho-xylene and meta-xylene may be subject to isomerization. The isomerized aromatic stream 88 may be fractionated in a second xylene fractionation unit 96 to recover xylenes in a second xylene fractionation C7+ stream 98 that includes xylenes and a second xylene fractionation overhead stream 102. The second xylene fractionation C7+ stream 98 may be returned to xylene separation, such as in the xylene column 59, to be further processed for xylene recovery along with the heavy reformate stream 12. In an embodiment, the second xylene fractionation C7+ stream 98 is returned to xylene separation in the absence of olefin reduction or clay treatment prior to separating xylenes from the second xylene fractionation C7+ stream 98. In another embodiment, the meta-xylene raffinate stream 90 is isomerized in the presence of an ethylbenzene isomerization catalyst. In this embodiment, the second xylene fractionation C7+ stream 98 may be clay treated, such as in a first clay unit 46, prior to separating xylenes from the second xylene fractionation C7+ stream 98.

The C9+ stream 63 may be further fractionated in an A9/A10 fractionation column 90, which is in fluid communication with the xylene column 59. The “A9/A10 fractionation column”, as referred to herein, is a fractionation column operated under conditions that are effective to separate compounds that have 11 or more carbon atoms from compounds that have less than 11 carbon atoms. In particular, the C9+ stream 63 may be fractionated into a C9 aromatic stream 92 that primarily includes compounds having 9 and 10 carbon atoms, e.g., at least 50 weight % of compounds that have 9 or 10 carbon atoms, and an A11+ fraction 94 that primarily includes compounds having at least 11 carbon atoms, e.g., at least 50 weight % of compounds that have at least 11 carbon atoms.

As set forth above, the reformate stream 14 is fractionated into the heavy reformate stream 12 and the light reformate overhead stream 16. The light reformate overhead stream 16 includes aromatic compounds and non-aromatic compounds, and the aromatic compounds may be separated from the non-aromatic compounds in the light reformate overhead stream 16 to produce a reformate aromatic stream 60 that includes the aromatic compounds and a reformate raffinate stream 62 that includes the non-aromatic compounds. The reformate raffinate stream 62 may be provided as a product stream or for other industrial processes, while the reformate aromatic stream 60 may be subject to further separation to recover benzene as described in further detail below.

Aromatic compounds and non-aromatic compounds may be difficult to separate through conventional fractionation due to similar boiling points, although various extraction techniques are known in the art for separating aromatics from non-aromatics. Examples of suitable extraction techniques that may be employed include, but are not limited to, azeotropic distillation, extractive distillation, and liquid/liquid solvent extraction. In an embodiment, a separation unit 64 is in fluid communication with the reformate splitter 58 for receiving the light reformate overhead stream 16 and for separating the light reformate overhead stream 16 into the reformate aromatic stream 60 and the reformate raffinate stream 62. As one example, the separation unit 64 may be an extraction unit 64 that operates through liquid/liquid solvent extraction using an appropriate solvent to effectuate separation of the aromatic compounds from the non-aromatic compounds. One specific example of a suitable extraction unit 64 is a sulfolane extraction unit 64 that operates through liquid phase extraction using sulfolane as the solvent to effectuate separation of the aromatic compounds from the non-aromatic compounds in the light reformate overhead stream 16.

Depending upon desired end uses for the reformate raffinate stream 62, further processing of the reformate raffinate stream 62 may be conducted in accordance with the processes described herein. For example, in an embodiment the reformate raffinate stream 62 may be provided to an ethylene cracking unit (not shown), where an olefin restriction is generally in place on the feed to the ethylene cracking unit. Thus, unsaturated species in the reformate raffinate stream 62 may be selectively hydrogenated to produce a reformate product stream 67 that has less unsaturated species than the reformate raffinate stream 62, thereby making the reformate product stream 67 suitable for use as a feed in the ethylene cracking unit. To effectuate selective hydrogenation and as shown in FIG. 1, a second ORP unit 48 may be in fluid communication with the separation unit 64 to receive the reformate raffinate stream 62. In other embodiments, such as under conditions where the reformate raffinate stream 62 is provided to a gasoline pool, selective hydrogenation is omitted to avoid degrading the reformate raffinate stream 62.

Aromatic compounds in the reformate aromatic stream 60 may be further separated to recover the various aromatic compounds through conventional techniques to yield separate benzene, xylene, and, if desired, toluene fractions. Alternatively, toluene may be further converted to yield additional benzene and xylenes therefrom, as described in further detail below. Separation of the aromatic compounds from the reformate aromatic stream 60 is described in further detail below.

The crude toluene stream 32 is converted in the presence of a catalyst that includes acid function and metal function to produce a converted aromatic stream 38. In particular, the crude toluene stream 32 may be combined with the C9 aromatic stream 92 and with a toluene fraction 76 that is separated from the reformate aromatic stream 60 to form a conversion feed 36 that is subject to conversion. Because the catalyst that includes acid function and metal function is used, prior treatment of olefins and alkenyl benzene before conversion is unnecessary. Whereas ORP units or clay units may conventionally be used to treat olefins and alkenyl benzene from hydrocarbon streams that are subject to conversion, the conversion feed 36 may be converted in the absence of prior treatment of olefins and alkenyl benzene therein. It is to be appreciated that the crude toluene stream 32 is generally depleted of olefins and alkenyl benzene due to prior olefin reduction processing prior to separation of the hydrogenated xylene stream 40. However, the olefins and alkenyl benzene in the mixed xylene stream 61 are treated due to detrimental impact of the presence of olefins and alkenyl benzene on separation of the xylenes and not due to conversion requirements. Because olefins and alkenyl benzene may be present in the conversion feed 36, unnecessary treatment of olefins and alkenyl benzene from the toluene fraction 76 and the C9 aromatic stream 92 is avoided, thereby maximizing process efficiency while minimizing associated costs of extra ORP units or clay units.

Referring to FIG. 1, a catalyzing unit 42 may be disposed in fluid communication with the adsorption unit 80 for transalkylating the crude toluene stream 32 in the presence of the catalyst that includes acid function and metal function to produce a transalkylated aromatic stream 38. The catalyzing unit 42 may further be in fluid communication with the toluene column 74 and with the A9/A10 fractionation column 90, with the crude toluene stream 32, the toluene fraction 76, and the C9 aromatic stream 92 combined prior to the catalyzing unit 42 to form the transalkylation feed 36. Transalkylation generally involves conversion of multiple alkylated aromatic compounds to primary monoalkylated aromatic compounds such as toluene and xylenes in the presence of the catalyst that includes acid function and metal function under transalkylating conditions. Whereas clay treatment of styrene (which is generally separated with C7 aromatic compounds including toluene) results in compounds that are ultimately separated from the process and contribute to loss of yield, no prior treatment of olefins and alkenyl benzene generally occurs prior to transalkylation, save for treatment of olefins and alkenyl benzene in the mixed xylene stream 61 that ultimately supplies the crude toluene stream 32. As such, olefins and alkenyl benzene may be present in the transalkylation feed 36. Instead of employing conventional transalkylation catalysts, the catalysts that include acid function and metal function are used due to the presence of olefins and alkenyl benzene in the transalkylation feed 36 to the catalyzing unit 42. Suitable catalysts that include acid function and metal function may include a zeolite component, an acid promoted alumina, or the like. The zeolite component may be a pentasil zeolite, which include the structures of MFI, MEL, MTW, MTT and FER (IUPAC Commission on Zeolite Nomenclature), a beta zeolite, a mordenite, or an alternative structure with similar activity. The metal function may be provided, for example, by a noble metal and/or a base metal. Examples of suitable noble metals include platinum-group metals chosen from platinum, palladium, rhodium, ruthenium, osmium, and iridium. Examples of suitable base metals include those chosen from rhenium, tin, germanium, lead, cobalt, nickel, molybdenum, indium, gallium, zinc, uranium, dysprosium, thallium, and mixtures thereof The base metal may be combined with another base metal, or with a noble metal. The metal component may be present in the metallic, oxide, sulfide or other catalytically active form. Suitable metal amounts in the catalyst that includes acid function and metal function may be from about 0.01 to about 10 weight %, such as from about 0.1 to about 3 weight %, or such as from about 0.1 to about 1 weight %. Suitable zeolite amounts in the catalyst that includes acid function and metal function may be from about 1 to about 99 weight %, such as from about 10 to about 90 weight %, or such as from about 25 to about 75 weight %. The balance of the catalyst that includes acid function and metal function may be an inorganic oxide binder. The catalysts that include acid function and metal function may be employed under conventional transalkylation conditions.

The transalkylated aromatic stream 38 includes aromatic compounds that may be further separated to recover the various aromatic compounds. In an embodiment and as shown in FIG. 1, the reformate aromatic stream 60 and the transalkylated aromatic stream 38 from the catalyzing unit 42 are combined to form a combined aromatics stream 66, which may then be subject to conventional aromatics separation. Optionally, olefins and alkenyl benzene in the combined aromatics stream 66 are treated, such as through ORP or clay treatment. For example and as shown in FIG. 1, a second clay unit 49 may be in fluid communication with the catalyzing unit 42, and optionally further in fluid communication with the separation unit 64, for treating the olefins and alkenyl benzene therein. In an embodiment and as shown in FIG. 1, a benzene column 68 is in fluid communication with the catalyzing unit 42 and with the separation unit 64 for receiving the combined aromatics stream 66, optionally with the second clay unit 49 disposed therebetween. The combined aromatics stream 66 is fractionated into a benzene fraction 70 and a C7+ stream 72 within the benzene column 68. The benzene fraction 70 includes primarily benzene, e.g., at least 50 weight % benzene, although higher purity benzene is generally obtained in the benzene fraction 70. The C7+ stream 72 primarily includes compounds that have at least 7 carbon atoms, e.g., at least 50 weight % of compounds that have at least 7 carbon atoms. The benzene fraction 70 may be taken as a product stream or used in other industrial processes. A toluene column 74 is in fluid communication with the benzene column 68 for receiving the C7+ stream 72, and the C7+ stream 72 is fractionated within the toluene column 74 into a toluene fraction 76 and a C8+ fraction 78 that includes compounds having at least 8 carbon atoms such as, for example, xylenes and C9 and C10+ aromatic compounds. In an embodiment and as shown in FIG. 1, and as also alluded to above, the toluene fraction 76 is returned to the catalyzing unit 42 for conversion into benzene and xylenes through disproportionation and transalkylation, although it is to be appreciated that in other embodiments the toluene fraction 76 may be taken as a product stream or used in other industrial processes. The C8+ fraction 78 may be combined with the heavy reformate stream 12 to be further processed for xylene recovery.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention. It being understood that various changes may be made in the function and arrangement of elements described in an exemplary embodiment without departing from the scope of the invention as set forth in the appended claims.

What is claimed is:

1. A process for preparing aromatic compounds, the process comprising the steps of:
providing a heavy reformate stream comprising C7 hydrocarbons, xylenes, styrene, and compounds having more than 8 carbon atoms;
separating xylenes and styrene from the compounds having more than 8 carbon atoms in the heavy reformate stream to form a mixed xylene stream comprising the xylenes and styrene and a C9+ stream comprising compounds with less volatility than C8 aromatics;
selectively hydrogenating the styrene within the mixed xylene stream to form a hydrogenated xylene stream comprising xylenes and ethylbenzene; and
separating at least some of the xylenes from the ethylbenzene in the hydrogenated xylene stream to form a C8 raffinate stream comprising the ethylbenzene and a xylene product stream comprising one or more xylene species.

2. The process of claim 1, wherein selectively hydrogenating the styrene within the mixed xylene stream comprises selectively hydrogenating the styrene within the mixed xylene stream immediately prior to separating xylenes from the ethylbenzene in the hydrogenated xylene stream.

3. The process of claim 1, further comprising fractionating a reformate stream into the heavy reformate stream and a light reformate overhead stream comprising compounds having higher volatility than xylene.

4. The process of claim 3, further comprising separating aromatic compounds from non-aromatic compounds in the light reformate overhead stream to produce a reformate aromatic stream and a reformate raffinate stream.

5. The process of claim 4, further comprising selectively hydrogenating unsaturated species in the reformate raffinate stream to produce a reformate product stream.

6. The process of claim 3, further comprising reforming a hydrotreated stream to produce the reformate stream.

7. The process of claim 6, further comprising hydrotreating a naphtha feed stream to form the hydrotreated stream.

8. The process of claim 1, wherein separating xylenes from the ethylbenzene in the hydrogenated xylene stream comprises separating the hydrogenated xylene stream into the xylene product stream wherein the xylene product stream comprises para-xylene, a meta-xylene product stream comprising meta-xylene, and a meta-xylene raffinate stream comprising ethylbenzene.

9. The process of claim 8, wherein separating the xylenes from the ethylbenzene in the hydrogenated xylene stream comprises separating the hydrogenated xylene stream into the xylene product stream and the C8 raffinate stream, and further comprises separating the C8 raffinate stream into the meta-xylene product stream comprising meta-xylene and the meta-xylene raffinate stream.

10. The process of claim 9, further comprising isomerizing the meta-xylene raffinate stream to produce an isomerized aromatic stream stream.

11. The process of claim 10, further comprising fractionating the isomerized aromatic stream into a second xylene fractionation C7+ stream and a second xylene fractionation overhead stream.

12. The process of claim 11, further comprising returning the second xylene fractionation C7+ stream to xylene separation with the heavy reformate stream in the absence of olefin reduction or clay treatment prior to separating xylenes from the second xylene fractionation C7+ stream.

13. The process of claim 11, wherein isomerizing the meta-xylene raffinate stream comprises isomerizing the meta-xylene raffinate stream in the presence of an ethylbenzene isomerization catalyst, and wherein the process further comprises clay treating the second xylene fractionation C7+ stream.

14. The process of claim 1, further comprising separating the C9+ stream into a C9 aromatic stream comprising compounds having 9 carbon atoms and a C10+ stream comprising compounds having at least 10 carbon atoms.

15. The process of claim 14, further comprising returning the C9 aromatic stream to transalkylation.

16. The process of claim 1, wherein separating xylenes and styrene from the compounds having more than 8 carbon atoms in the heavy reformate stream comprises separating the heavy reformate stream in the absence of selective hydrogenation of the heavy reformate stream.

17. A process for preparing aromatic compounds, the process comprising:
hydrotreating a naphtha feed stream to form a hydrotreated stream;
reforming the hydrotreated stream to produce a reformate stream;
fractionating the reformate stream into a heavy reformate stream comprising C7 hydrocarbons, xylenes, styrene, and compounds having more than 8 carbon atoms and a light reformate overhead stream comprising compounds having higher volatility than xylene;
separating aromatic compounds from non-aromatic compounds in the light reformate overhead stream to produce a reformate aromatic stream and a reformate raffinate stream;
separating xylenes and styrene from the compounds having more than 8 carbon atoms in the heavy reformate stream to form a mixed xylene stream comprising the xylenes and styrene and a C9+ stream comprising compounds with less volatility than C8 aromatics;
selectively hydrogenating the styrene within the mixed xylene stream to form a hydrogenated xylene stream comprising xylenes and ethylbenzene; and
separating the hydrogenated xylene stream into a xylene product stream comprising para-xylene, a meta-xylene product stream comprising meta-xylene, and a meta-xylene raffinate stream comprising ethylbenzene.

18. An apparatus for preparing aromatic compounds, wherein the apparatus comprises:
a hydrotreating unit for receiving a naphtha feed stream and for hydrotreating the naphtha feed stream to form a hydrotreated stream;
a reforming unit in fluid communication with the hydrotreating unit for receiving the hydrotreated stream and for reforming the hydrotreated stream to produce a reformate stream;
a reformate splitter in fluid communication with the reforming unit for receiving the reformate stream and for fractionating the reformate stream into a heavy reformate stream comprising C7 hydrocarbons, xylenes, styrene, and compounds having more than 8 carbon atoms and a light reformate overhead stream comprising compounds having higher volatility than xylene;
a xylene column in fluid communication with the reformate splitter for receiving and for separating the heavy reformate stream to produce a mixed xylene stream comprising xylenes and styrene and a C9+ stream comprising compounds with less volatility than C8 aromatics;
an olefin reduction processing unit in fluid communication with the xylene column for receiving the mixed xylene stream and for selectively hydrogenating the styrene within the mixed xylene stream to form a hydrogenated xylene stream comprising xylenes and ethylbenzene; and
an adsorption unit in fluid communication with the olefin reduction processing unit for receiving the hydrogenated xylene stream and for separating xylenes from the ethylbenzene in the hydrogenated xylene stream to form a C8 raffinate stream comprising the ethylbenzene and a xylene product stream comprising one or more xylene species. an isomerizing unit in fluid communication with the adsorption unit for isomerizing the ethylbenzene from the C8 raffinate stream in the presence of an isomerization catalyst to produce an isomerized aromatic stream comprising xylenes.

19. The apparatus of claim 18, free from a clay unit disposed between the reformate splitter and the adsorption unit.

20. The apparatus of claim 18, further comprising a clay unit in fluid communication with the catalyzing unit for receiving the isomerized aromatic stream and for treating olefins and alkenyl benzene in the isomerized aromatic stream.
